(19) United States
(12) Patent Application Publication
(10) Pub. No.: US 2009/0209036 A1
(43) Pub. Date: Aug. 20, 2009

(54) Method for Accelerating Somatic Mutations and use Thereof in Proteomics

(75) Inventors: Claude-Agnes Reynaud, Paris (FR); Jean-Claude Weill, Paris (FR)

Correspondence Address: Larson & Anderson, LLC, P.O. BOX 4928, DILLON, CO, 80435, US

(73) Assignee: INSTITUT NECKER, Paris (FR)

(21) Appl. No.: 11/574,997

(22) PCT Filed: Sep. 12, 2005
(86) PCT No.: PCT/FR05/02260

§ 371 Date: May 29, 2008

(30) Foreign Application Priority Data
Sep. 10, 2004 (FR) 0409605

(51) Int. Cl. C12N 15/74; C07H 21/00

(52) U.S. Cl. 435/455; 536/23.2

(57) ABSTRACT

The invention relates to a method of accelerating the induction of somatic mutations in vitro. The inventive method comprises the expression of at least one cDNA expressing a modified version of the AID gene in the cells to be mutated, in culture conditions and a medium that are suited thereto, said modified version resulting from an AID gene in which the three hydrophobic amino acids, leu189, phe193 and leu196, have been replaced by means of alanine mutations in each case. The invention can be used to induce mutations in Burkitt's lymphoma BL2. The invention can also be used to induce mutations in the immunoglobulin genes of immortalised antibody-producing cells, such as mouse hybridoma cells, human hybridoma cells or human B-cell lines immortalised by the Epstein-Barr virus (EBV).

### FIELD OF THE INVENTION

The present invention relates to biology, and more particularly to the field of adaptation by directed mutation, specific to the living world. More particularly, it relates to accelerating the induction of somatic mutations in vitro and to the applications made possible by improvements to such a technique.

In a particular embodiment, the invention relates to the induction of mutations in BL2 Burkitt's lymphoma.

In another aspect, the invention relates to the induction of mutations in immortalized antibody-producing cells, i.e. mouse hybridomas or human hybridomas or human B-cell lines immortalized with Epstein-Barr virus (EBV).

### TECHNICAL BACKGROUND OF THE INVENTION

For the sake of simplicity, BL2 Burkitt's lymphoma is referred to below. However, it should be emphasized that this is merely in order to facilitate the explanation of the technique in question and the inventive concept claimed, without thereby limiting the invention to this aspect.

The Type I BL2 Burkitt's lymphoma cell line has the characteristics of centroblastic B cells. This cell line is the closest immortalized counterpart to germinal center centroblasts. Its germinal center origin has been confirmed by the presence of somatic mutations in the V_{H} immunoglobulin genes of Burkitt's lymphomas.

The homogeneity and stability of BL2 cells in suitable cultures has been pointed out (S. Denépoux et al., Immunity, Vol. 6, 35-46, 1997). It has been proposed, in keeping with the centroblast phenotype, that BL2 be considered to result from the transformation of a germinal center centroblast that has undergone several somatic mutation phases.

Moreover, B-cells are considered to undergo hypermutation at the centroblast stage (V. Pascual et al., J. Exp. Med. 180, 329-339 (1994)).

The immunoglobulin hypermutation phenomenon takes place in the germinal centers, after stimulation of a B-cell with a T-dependent antigen.

In particular, S. Denépoux et al. (op. cit.) established that lymphoma cells, particularly BL2 cells, can trigger a hypermutation process after aggregation of their surface receptor and co-culture with an auxiliary or amplifying T-cell, after one week of culture. It was concluded in this article that through repeated activations of BL2 cells, it is possible to increase the mutation frequency. In addition, these authors emphasized that mutations induced in vitro in BL2 do not affect the constant region of the IgM and do not cause the appearance of an antigen-directed selection.

Furthermore, J. Yélamos et al., Nature, Vol. 376, pp. 225-229 (1995) reported on experiments which tended to show that the V gene fragment of immunoglobulin is not essential for hypermutation and that the construction of artificial mutation substrates could therefore be simplified.

The BL2 line, derived from a Burkitt's lymphoma, can thus be used as a targeted mutagenesis tool for altering a gene of interest.

Despite these developments in the prior art, there are no effective means available for studying these phenomena and obtaining practical results from them, particularly for purposes of diagnostics and/or follow-up and the treatment of tumoral processes, with satisfactory practicability and speed.

There is therefore a need for means that make it possible to perform fast and reliable somatic hypermutation tests. More particularly, there is a need for means that provide a somatic mutation induction model applicable to V genes but also to other genes that could be involved in a tumoral process, especially in humans, such as for example the Bcl-6 and Fas Ligand (FasL) genes.

The present applicant has previously demonstrated that immunoglobulin gene mutation can be induced by signals that imitate the initiation of the immunoglobulin gene hypermutation process: stimulation with an anti-IgM antibody, plus co-culture with auxiliary T-cells, or stimulation with anti-CD19 and anti-CD21 antibodies plus biotinylated anti-IgM antibodies, which can then be aggregated using magnetic beads coupled with streptavidin.

The present inventors have also previously demonstrated that it is possible to induce homologous recombination in this line, although the use of this technique is normally limited to ES (embryonic stem cell) lines or to the line derived from the avian lymphoma DT40.

### SUMMARY OF THE INVENTION

We have now found a method for inducing somatic mutations wherein said mutations are accelerated, particularly in the BL2 line derived from Burkitt's lymphoma, by expressing cDNAs which themselves express modified versions of the AID gene (the abbreviation AID stands for “activation-induced cytidine deaminase”). To this end, we developed specially adapted, AID-modified cDNAs, which are also part of the present invention.

### BRIEF DESCRIPTION OF THE DRAWINGS

FIG. 1 represents the natural nucleotide coding sequence of human AID and the peptide encoded by said sequence.

FIG. 2 is a representation of the respective proportions of VH sequences in the BL2 line having a given number of mutations, respectively in cells transfected with wild-type AID and in cells transfected with the mutant AID (SEQ ID NO.1 according to the present invention).

### DETAILED DESCRIPTION OF THE INVENTION

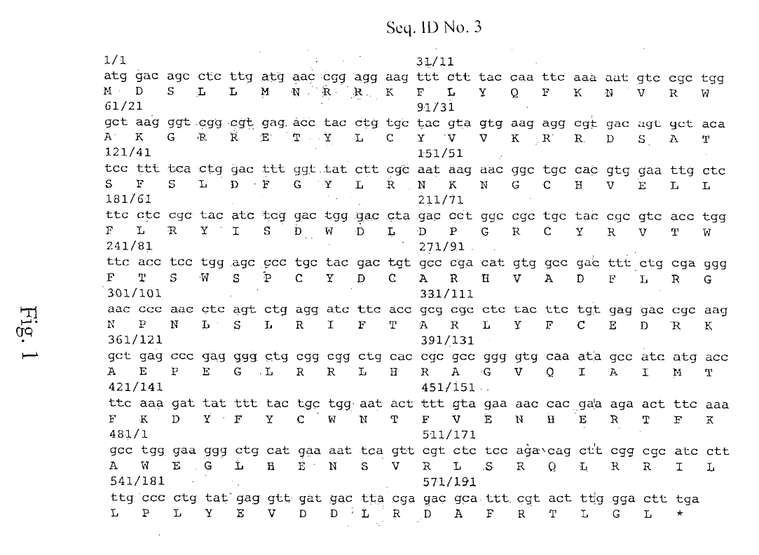

The AID gene (see FIG. 1) is responsible for initiating the hypermutation process by deaminating the cytidines of the immunoglobulin V gene into uracils, which results in a reparation or an erroneous replication of these abnormal bases in the DNA.

AID is an essentially cytoplasmic protein which must be targeted into the nucleus of a cell in order to exert its deamination activity. Yet AID is constantly shuttling between nucleus and cytoplasm (see, for example, S. Ito et al., PNAS, Feb. 17, 2004, Vol. 101, No. 7, pp. 1975-1980), and its cytoplasmic localization results from an active export from the nucleus, performed by the CRM1 protein (see for example K. M. McBride et al., J. Exp. Med., Vol, 199, No. 9, May 3, 2004, pp.1235-1244). The latter identifies a consensus site, present in the AID protein.

We have now found that it is possible to interrupt this export and to obtain an essentially nuclear localization of the AID protein by mutating three hydrophobic amino acids in this consensus site of the AID protein, i.e., leu189, phe193 and leu196, into alanine.

Thus, we found experimentally that the mutagenesis observed at the immunoglobulin locus is increased constituently by a factor of 5 to 10 relative to the mutation rate that would be obtained, all things being equal, using the known techniques mentioned above.

The nucleotide coding sequence of human AID carrying the mutation indicated above at the appropriate positions is represented, along with the peptide encoded by said sequence, by the sequence SEQ ID NO.1, comprising the mutations of the three amino acids leu189, phe193 and leu196 into alanine in the sequence of the AID.

This mutagenesis is performed using methods well known to the person skilled in the art.

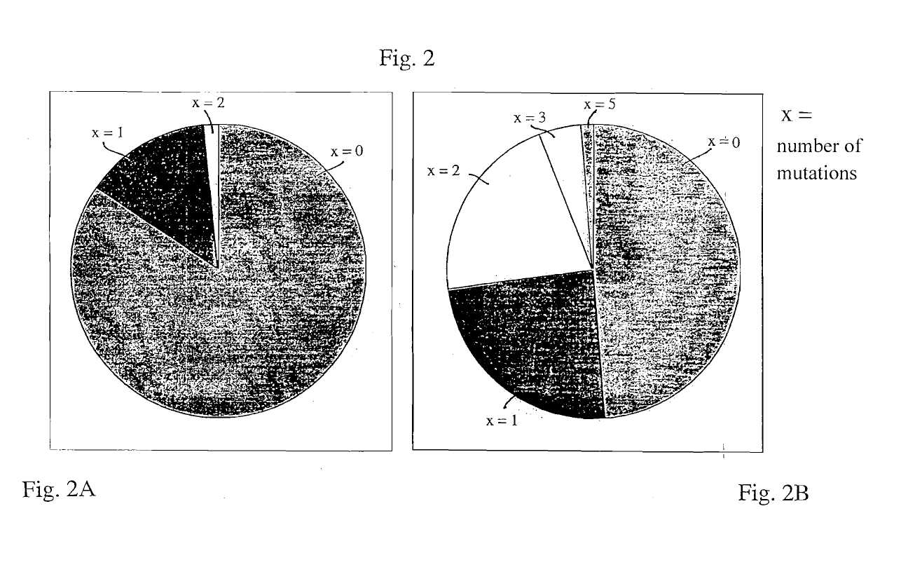

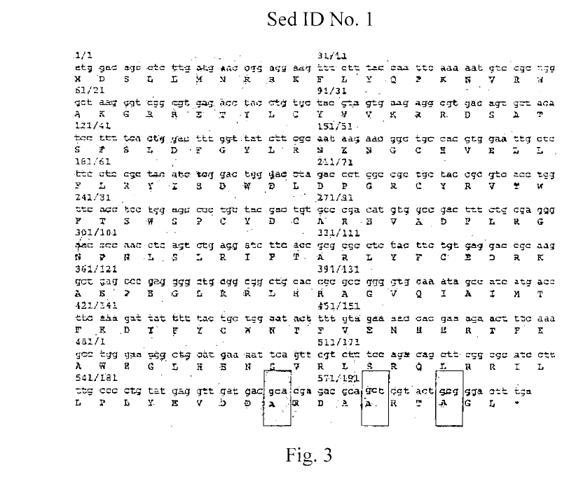

The effect obtained by the method according to the invention is confirmed by the results of experiments (performed conventionally, in keeping with practices known to the person skilled in the art) summarized in the diagrams of the attached FIGS. 2A-B.

We thus found that lymphoma cells, particularly BL2 cells, can trigger a sharply accelerated hypermutation process in their immunoglobulin genes, contrary to the data of the prior art, according to which a comparable process would be long and tedious to implement, or would require other techniques to obtain a slightly accelerated mutation. Moreover, the method according to the invention produces a remarkable distribution of the number of mutations obtained (see FIG. 2B).

The mutation frequency (per 100 base pairs) was found to be 0.22% for cells transfected with the mutant AID gene according to the invention having only the 3 amino acids 189, 193 and 196 replaced by alanine (i.e. SEQ ID NO.1), as compared to only 0.05% for cells transfected with the wild-type human AID according to the sequence represented in FIG. 1.

More precisely, we have now unexpectedly found that, with the BL2 cell as a model, it is possible to obtain in vitro, with quite exceptional speed and efficiency, the induction of somatic mutations in appropriate cells by transfecting the cells to be mutated with at least one mutant AID gene as described above and represented by SEQ ID NO.1 with SEQ ID NO.2 in the sequence listing representing the peptide encoded by said sequence.

Thus, the first subject of the invention is a method for accelerating the induction of somatic mutations in vitro or ex vivo, comprising (1) the addition to the cells to be mutated, under culture conditions and in a medium suitable for said cells, of a cDNA expressing a modified version of the AID gene wherein the three hydrophobic amino acids leu189, phe193 and leu196 have been replaced, in each case by mutation into alanine.

According to one characteristic, the induction of somatic mutations according to the invention is carried out over a period of at least 7 days, and more preferably over a period of about 8 days.

According to an advantageous characteristic, the method according to the invention does not include the use of co-cultures with cells of another cell type, particularly with a T cell.

According to a specific characteristic, the method according to the invention is used to induce mutations in BL2 Burkitt's lymphoma.

Another subject of the invention is the use of this method for inducing somatic mutations to perform in vitro hypermutagenicity tests at the protein and/or gene level.

The practical modalities best suited to the use of the method for accelerating somatic mutations according to the invention are well known to the person skilled in the art, who has access to routine and/or testing means for adjusting the parameters, as necessary.

In a first aspect of the invention, this use is applied to B lymphomas, particularly to human B lymphomas, more specifically for the purpose of inducing mutations in BL2 Burkitt's lymphoma.

In another aspect of the invention, this use is applied to other immortalized antibody-producing cells, i.e. mouse hybridomas or human B cell lines immortalized with Epstein-Barr virus (EBV).

Another subject of the invention is the use of the method according to the invention to induce somatic mutations in the immunoglobulin genes of immortalized antibody-producing cells, particularly those chosen from among mouse hybridomas, human hybridomas, and human B-cell lines immortalized with Epstein-Barr virus. Techniques similar to those recommended for Burkitt's lymphomas are used for this purpose.

In practice, in order to induce somatic mutations in vitro, particularly in the BL2 cell according to the invention, a cell transfection is performed using techniques that are well known to the person skilled in the art.

Another subject of the invention is a kit for inducing somatic mutations, characterized in that it comprises a system inducing accelerated somatic mutations as defined above.

Another subject of the invention is the use of this kit for the qualitative and/or quantitative identification of components of the mutasome, particularly by protein analysis.

In one embodiment, the use of the kit according to the invention includes the identification of the post-translational modifications induced, particularly by identifying, isolating and analyzing a component of the mutasome, particularly a protein component, that appears during said induction.

According to a characteristic of a preferred implementation, this use includes providing at least one gene, particularly a coding gene for a protein of interest, in which one wishes to induce mutations while said gene is included in a cassette containing the promoter and the enhancer (the enhancer of the genes coding for the heavy or light chain of the IgGs), and while said gene is transfected into lymphoma cells, particularly BL2 Burkitt's lymphoma cells.

According to an advantageous embodiment, in order to mutate any sequence, the sequence to be mutated is flanked by the promoter and the enhancer of the Ig, preferably in a mutation cassette.

A person skilled in the art, based on the above information and his/her own knowledge, without going beyond the scope of the present invention, could thus conceive of similar or equivalent test systems and various concrete uses, which may or may not be related to those described herein.

1-16. (canceled)

17. Method for induction of somatic mutations in vitro, comprising the step of expressing in the cells to be mutated, under culture conditions and in a medium suitable for said cells, cDNA encoding a mutant form of an activation-induced cytidine deaminase (AID) gene (SEQ ID NO. 3) thereby inducing somatic mutations in the cells, wherein the three hydrophobic amino acids leu189, phe193 and leu196 have been replaced by mutation into alanine.

18. Method according to claim 17, wherein in order to obtain the in vitro induction of somatic mutations in appropriate cells, the cells to be mutated are transfected with the mutant form of the AID gene.

19. Method according to claim 17, wherein the mutant form of the AID gene has the sequence given in SEQ ID NO. 1.

20. Method according claim 19, wherein the induction of mutations is carried out over a period of at least 7 days.

21. Method according claim 18. wherein the induction of mutations is carried out over a period of at least 7 days.

22. Method according claim 17 wherein the induction of mutations is carried out over a period of at least 7 days.

23. Method of claim 17, wherein the cells are B lymphoma cells.

24. Method of claim 23, wherein the cells are human B lymphoma cells.

25. Method of claim 24, wherein the cells are BL2 Burkitt's Lymphoma cells.

26. Method according to claim 24, wherein in order to obtain the in vitro induction of somatic mutations in appropriate cells, the cells to he mutated are transfected with the mutant form of the AID gene.

27. Method according to claim 24, wherein the mutant form of the AID gene has the sequence given in SEQ ID NO. 1.

28. Method according claim 27 wherein the induction of mutations is carried out over a period of at least 7 days.

29. Method according claim 26 wherein the induction of mutations is carried out over a period of at least 7 days.

30. Method according claim 24 wherein the induction of mutations is carried out over a period of at least 7 days.

31. Method of claim 17, wherein the cells are immortalized antibody-producing cells.

32. Method of claim 31, where the immortalized antibody-producing, cells are selected from the group consisting of mouse hybridomas, human hybridomas and human B cell limes immortalized with Epstein-Ban- virus.

33. Method according to claim 32, wherein the mutant form of the AID gene has the sequence given in SEQ ID NO. 1.

34. Method according to claim 31, wherein the mutant form of the AID gene has the sequence given in SEQ ID NO. 1.

35. A polynucleotide comprising a sequence encoding a mutant form of human activation-induced cytidine deaminase (AID) gene (SEQ ID NO. 3) wherein the bases encoding three hydrophobic amino acids leu189, phe193 and leu196 have been replaced by mutation into codons encoding into alanine.

36. The polynucleotide of claim 35, wherein the sequence encoding a mutant form of human AID comprises SEQ ID NO: 1.


SEQUENCE LISTING (2 sequences)

SEQ ID NO: 1 - DNA, 597 bp, Artificial sequence
  modified human sequence for AID
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacgcacgagacgcagctcgtactgcgggactttga

SEQ ID NO: 2 - protein, 198 aa, Artificial sequence
  modified human sequence for AID
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDARDAARTAGL